(19) United States
(12) Patent Application Publication
(10) Pub. No.: US 2020/0352847 A1
(43) Pub. Date: Nov. 12, 2020

(54) SPECIAL POLYURETHANE UREA FOR INCREASING THE WATER RESISTANCE OF A SHAPED HAIRSTYLE

(71) Applicant: Covestro Deutschland AG, Leverkusen (DE)

(72) Inventors: Sophie Viala, Köln (DE); Laurence Pottie, Köln (DE)

(21) Appl. No.: 16/764,485

(22) PCT Filed: Dec. 17, 2018
(86) PCT No.: PCT/EP2018/085170

§ 371 Date: May 15, 2020

(30) Foreign Application Priority Data
Dec. 18, 2017 (EP) 17208264.6

(51) Int. Cl. A61K 8/87; A45D 7/04

(52) U.S. Cl. CPC A61K 8/87; A45D 7/04

(57) ABSTRACT

The invention relates to the use of a polyurethane urea on hair for improving the water resistance of the hairstyle obtained by shaping the hair, which polyurethane urea is obtained by reacting at least a) a polyisocyanate component, b) a polymer polyol component, c) a hydrophilic component, and d) an amino functional chain extender component, wherein the polyisocyanate component comprises a) ≥75% mol-% isophorone diamine (IPDI) and the amino functional chain extender component comprises c) ≥75 mol-% isophorone diamine (IPDA).

The invention relates to the use of a special polyurethane urea on hair to improve the water resistance of the hairstyle obtained by shaping the hair, to a method for water-resistant and/or washable shaping of hair using the special polyurethane urea, and to a composition comprising the special polyurethane urea in particular presentations.

Products known as hair fixatives are used for styling and stabilizing a variety of hairstyles. Hair fixatives are usually in the form of mousses or hairsprays of barely differing composition. Mousses are applied to damp hair as an aid for modeling the hairstyle. By way of contrast, hairsprays, hair creams, hair gels or hair waxes are applied in air to dry, already-styled hair to fix the hairstyle for everyday wear.

In the case of hairsprays and mousses, the means of fixing or styling the hairstyle usually takes the form of aerosol containers, squeeze bottles or preparations sprayable by a pumping, spraying or foaming devices, which consist of an alcoholic, aqueous or aqueous-alcoholic solution of film-forming natural or synthetic polymers. These polymers may be selected from the group of nonionic, cationic, amphoteric or anionic polymers.

Acrylate-based anionic or amphoteric polymers are commonly used as film-forming polymers in the prior art. The use of polyurethanes and polyurethane ureas as film formers is, however, also known. For example, WO 2009/118105 A1 describes hair-fixative compositions that obtain a polyurethane urea obtainable by reacting a water-insoluble, non-water-dispersible, isocyanate-functional polyurethane prepolymer with an amino-functional compound. The hair-fixative compositions disclosed therein are well suited for stabilizing hairstyles until the next hair wash. They do not, however, give hairstyles lasting shape that would survive a hair wash.

It was therefore an object of the present invention to at least partly overcome one disadvantage of the prior art. A further object was to provide a polyurethane urea that, when used on hair, is able to achieve increased water resistance of the hairstyle shaped therewith. A particular object was to achieve water resistance lasting several washes of the hairstyle shaped with the polyurethane urea. A further object was to provide a method that allows a water-resistant and/or washable hairstyle to be created.

This object was surprisingly achieved by using a polyurethane urea obtainable by reacting at least the following components:
a) a polyisocyanate component,
b) a polymeric polyol component,
c) a hydrophilizing component, and
d) an amino-functional chain extender component,
   wherein the polyisocyanate component a) comprises >75 mol % of isophorone diisocyanate (IPDI) and the amino-functional chain extender component c) comprises >75 mol % of isophoronediamine (IPDA), on hair to improve the water resistance or washability of the hairstyle obtained by shaping the hair.

Water resistance in the context of the invention is understood as meaning that the resistance to water of the hairstyle shaped with the polyurethane urea is tested/determined. In these tests, the hair is at least partially covered with liquid water in a rinse treatment, that is to say soaked or rinsed therein. The water may be any form of liquid water that comes into contact with the hair in everyday life. Examples of this are drinking water, rainwater, swimming pool water (chlorinated, ozonized or kept microorganism-free by other means), seawater or distilled water.

Washability in the context of the invention is understood as meaning that the resistance to surfactant-containing water of the hairstyle shaped with the polyurethane urea is tested/determined, with the hair being completely covered by the surfactant-containing water during said washing treatment, that is to say rinsed therein.

An amino-functional chain extender component in the context of the invention is understood as meaning a component that comprises at least one compound having two isocyanate-reactive amino groups and no hydrophilizing groups.

In a preferred embodiment of the use of the polyurethane urea, the shaping of hair into a hairstyle is selected from the group consisting of:
a. straightening curly hair;
b. introducing curls into straight hair;
c. strengthening curls in already curled hair;
d. a combination of at least two of a. to c.

Straightening curly hair under a. may be done in any manner known to those skilled in the art, in particular by means of a straightening device. Curly hair is preferably straightened using a straightening iron, a straightening rod or a similar straightening device that is suitable for this purpose. The straightening device is preferably designed such that it is equipped with the aid of elements which may be heated to at least 150° C. The straightening device is preferably able to straighten curls to a degree of straightening of at least 50%, or preferably of at least 80%, or preferably of at least 90%. The degree of straightening is determined by measuring with a ruler the width of a tress of hair before and after straightening. A degree of straightening of 50%, 80% or 90% means that the width of the tress of hair after step a. has become respectively 50%, 80% or 90% narrower compared with the width before step a.

The introduction of curls into straight hair under b. may be done in any manner known to those skilled in the art, in particular by means of a curl-generating device. Straight hair is preferably made curly using a curling iron or a similar curl-generating device that is suitable for this purpose. The curl-generating device is preferably designed such that it includes with the aid of curl-generating elements which may preferably be gently heated, for example to 40 to 80° C. The curl-generating device is preferably able to create curls with a degree of curl of at least 50%, or preferably at least 80%, or preferably at least 90%. The degree of curl is determined by measuring with a ruler the length of a tress of hair before and after introducing the curls. A degree of curl of 50%, 80% or 90% means that the length of the tress of hair after step b. is respectively 50%, 80% or 90% shorter than before step b.

Strengthening curls under c. is preferably done in the same way as introducing curls under b. Alternatively or additionally, curls may be strengthened under c. by using a polyurethane-containing composition in preferably damp hair in the form of the use according to the invention, with the hair then being dried with or without aids. The curl-generating device is preferably able to create curls with a degree of curl of at least 30%, or preferably at least 40%, or preferably 50%. The degree of curl is determined by measuring with a ruler the length of a tress of hair before and after introducing the curls. A degree of curl of 30%, 40% or 50% means that the length of the tress of hair after step b. is respectively 30%, 40% or 50% shorter than before step c.

In a preferred embodiment of the use of the polyurethane urea, the shape of the styled hair is at least 5%, preferably at least 7%, or preferably at least 10%, retained after contact with water. The degree of preservation of the shape of the styled hair can preferably vary substantially here, depending on the type of treatment. Thus, it makes a difference whether the hair had been straightened before use according to the invention of the polyurethane urea or whether the hair had been curled. The degree of preservation of the shape of the styled hair is referred to hereinbelow also as “styling retention”.

In a preferred embodiment of the use of the polyurethane urea, the straightening of the hair under a. is at least 50%, or preferably at least 60%, or preferably at least at least 70%, or at least 80%, or at least 90%, retained after contact with water or the curling of the hair under b. is at least 5%, or preferably at least 7%, or preferably at least 10%, retained, or the curling under c. is at least 5%, or preferably at least 7%, or preferably at least 10%, retained.

The present invention therefore relates preferably to the use according to the invention, wherein the styling retention x for straightened hair, particularly in group a. referred to above, is at least 50%, or preferably at least 60%, or preferably at least 70%, or at least 80%, or at least 90%, after contact with water. The styling retention x_{straightened} for straightened hair is calculated according to:

$$Styling retention {x}_{straightened} = \frac{100 * [ Width before straightening - test width ]}{[ Width before straightening - width after straightening ]} ,$$

where the width before straightening, the width after straightening, and the test width are each expressed in cm and the test width is the width after the water-resistance test. The water-resistance test is preferably carried out by fully immersing straightened hair tresses individually in a 2-liter water bath heated to 38° C. for 30 seconds, allowing them to drip for 10 seconds, and allowing them to dry completely in air at room temperature for 24 h.

The present invention further preferably relates to the use according to the invention, wherein the styling retention x for curled hair, particularly in group b. and/or c. referred to above, is at least 5%, or preferably at least 7%, or preferably at least 10%. The styling retention x_{curled} for curled hair is calculated according to:

$$Styling retention {x}_{curled} = \frac{100 * [ Length before rolling - test length ]}{[ Length before rolling - length after rolling ]} ,$$

where the length before rolling, the length after rolling, and the test length are each expressed in cm and the test length is the length after the water-resistance test. The water-resistance test is carried out as described above, using rolled hair tresses instead of straightened hair tresses.

In a preferred embodiment of the use of the polyurethane urea, the polyurethane urea is in the form of a hair cosmetic composition.

In a preferred embodiment of the hair cosmetic composition, this is selected from the group consisting of a gel, a cream, an aerosol, a spray, a hair wax or a combination of at least two thereof.

In a preferred embodiment of the polyurethane urea for use according to the invention, the polyisocyanate component a) may comprise ≥80 mol %, preferably ≥85 mol %, more preferably 95 mol %, and particularly preferably 100 mol %, of IPDI.

Further polyisocyanates that can be used in component a) in addition to IPDI, in a molar proportion of less than 25 mol %, are aromatic, araliphatic, aliphatic or cycloaliphatic polyisocyanates having an NCO functionality of ≥2 that are known per se to those skilled in the art.

Examples of such polyisocyanates are butylene-1,4-diisocyanate, hexamethylene-1,6-diisocyanate (HDI), trimethylhexamethylene-2,2,4-diisocyanate and/or -2,4,4-diisocyanate, the isomeric bis(4,4′-isocyanatocyclohexyl)methanes or mixtures thereof in any desired isomer content, cyclohexylene-1,4-diisocyanate, phenylene-1,4-diisocyanate, tolylene-2,4-diisocyanate and/or -2,6-diisocyanate, naphthylene 1,5-diisocyanate, diphenylmethane 2,2′-diisocyanate and/or -2,4′-diisocyanate and/or -4,4′-diisocyanate, 1,3- and/or 1,4-bis(2-isocyanatoprop-2-yl)benzene (TMXDI), 1,3-bis(isocyanatomethyl)benzene (XDI), alkyl 2,6-diisocyanatohexanoate (lysine diisocyanates) having C1-C8 alkyl groups, and also 4-isocyanatomethyloctane 1,8-diisocyanate (nonane triisocyanate) and triphenylmethane 4,4′,4″-triisocyanate.

In addition to the polyisocyanates mentioned above, it is also possible to use proportions of modified diisocyanates or triisocyanates having uretdione, isocyanurate, urethane, allophanate, biuret, iminooxadiazinedione and/or oxadiazinetrione structures.

These are preferably polyisocyanates or polyisocyanate mixtures of the type mentioned above having exclusively aliphatically and/or cycloaliphatically bonded isocyanate groups and a mean NCO functionality in the mixture of 2 to 4, preferably 2 to 2.6, and more preferably 2 to 2.4.

If further polyisocyanates besides IPDI are used in component a), particular preference is given to using hexamethylene-1,6-diisocyanate, the isomeric bis(4,4′-isocyanatocyclohexyl)methanes, and mixtures thereof.

It is likewise preferable if the polymeric polyol component b) used a number-average molecular weights of ≥400 and ≤8000 g/mol, more preferably of 600 to 3000 g/mol, and/or has a mean OH functionality of 1.5 to 6, preferably of 1.8 to 3, and more preferably of 1.9 to 2.1.

Likewise advantageous is if the polymeric polyol component b) comprises or consists of a polyester, preferably a polyester based on adipic acid.

Possible constituents of the polymeric polyol component b) are the polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols, and polyester polycarbonate polyols known per se in polyurethane coatings technology. In b) these may be used individually or in any desired mixtures with one another.

Polyester polyols are, for example, the polycondensates of di- and optionally tri- and tetraols and di- and optionally tri- and tetracarboxylic acids or hydroxycarboxylic acids or lactones known per se. Instead of the free polycarboxylic acids, it is also possible to use the corresponding polycarboxylic anhydrides or corresponding polycarboxylic esters of lower alcohols for preparing the polyesters.

Examples of diols suitable for this purpose are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or the hydroxypivalate ester of neopentyl glycol, preference being given to hexane-1,6-diol and isomers, neopentyl glycol, and the hydroxypivalate ester of neopentyl glycol. In addition, it is also possible to use polyols such as trimethylolpropane, glycerol, erythritol, pentaerythritol, trimethylolbenzene or tris(hydroxyethyl) isocyanurate.

The dicarboxylic acids used may be phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. It is also possible to use the corresponding anhydrides as the acid source.

Provided the mean functionality of the polyol to be esterified is greater than 2, it is possible to additionally use monocarboxylic acids such as benzoic acid and hexanecarboxylic acid too.

Preferred acids are aliphatic or aromatic acids of the type mentioned above. Particular preference is given to adipic acid, isophthalic acid, and optionally trimellitic acid and very particular preference to adipic acid.

Examples of hydroxycarboxylic acids that may be used as co-reactants in the preparation of a polyester polyol having terminal hydroxyl groups include hydroxycaproic acid, hydroxybutyric acid, hydroxydecanoic acid, hydroxystearic acid and the like. Suitable lactones are caprolactone, butyrolactone and homologs. Preference is given to caprolactone.

In component b), it is also possible to use polycarbonates having hydroxyl groups, preferably polycarbonate diols, having number-average molecular weights Mn of 400 to 8000 g/mol, preferably of 600 to 3000 g/mol. These are obtainable by reacting carbonic acid derivatives, such as diphenyl carbonate, dimethyl carbonate or phosgene, with polyols, preferably diols.

Examples of such diols are ethylene glycol, propane-1,2- and 1,3-diol, butane-1,3- and 1,4-diol, hexane-1,6-diol, octane-1,8-diol, neopentyl glycol, 1,4-bis(hydroxymethyl)cyclohexane, 2-methylpropane-1,3-diol, 2,2,4-trimethylpentane-1,3-diol, dipropylene glycol, polypropylene glycols, dibutylene glycol, polybutylene glycols, bisphenol A, and lactone-modified diols of the type mentioned above. The polycarbonates having hydroxyl groups are preferably linear in structure.

Polyether polyols may likewise be used in component b). Suitable examples are the polytetramethylene glycol polyethers known per se in polyurethane chemistry such as are obtainable by cationic ring-opening polymerization of tetrahydrofuran.

Likewise suitable polyether polyols are the addition products of styrene oxide, ethylene oxide, propylene oxide, butylene oxides and/or epichlorohydrin on di- or polyfunctional starter molecules that are known per se.

Suitable starter molecules that may be used are all compounds known from the prior art, for example water, butyldiglycol, glycerol, diethylene glycol, trimethylolpropane, propylene glycol, sorbitol, ethylenediamine, triethanolamine, butane-1,4-diol. Preferred starter molecules are water, ethylene glycol, propylene glycol, butane-1,4-diol, diethylene glycol, and butyl diglycol.

In a further advantageous embodiment of the use according to the invention of the polyurethane urea, the hydrophilizing component c) is an anionically hydrophilizing component and preferably a sulfonate.

Suitable anionically or potentially anionically hydrophilizing compounds in component c) are compounds that have at least one isocyanate-reactive group, such as a hydroxyl or amino group, and at least one functionality such as —COO-M⁺, —SO₃-M⁺, —PO(O-M⁺)₂, where M⁺ is for example a metal cation, H⁺, NH₄⁺, NHR₃⁺, where R is in each case a C1-C12 alkyl radical, C5-C6 cycloalkyl radical and/or a C2-C4 hydroxyalkyl radical that enters into a pH-dependent dissociation equilibrium on interaction with aqueous media and may consequently be negatively charged or uncharged. Suitable anionically or potentially anionically hydrophilizing compounds are mono- and dihydroxycarboxylic acids, mono- and dihydroxysulfonic acids, and mono- and dihydroxyphosphonic acids, and salts thereof. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO3, as described in DE-A 2 446 440, pages 5-9, formula I-III. Preferred anionic or potentially anionic hydrophilization agents in component c) are those of the type mentioned above that have carboxylate/carboxylic acid groups and/or sulfonate groups.

Suitable nonionically hydrophilizing compounds in component c) are, for example, polyoxyalkylene ethers having at least one hydroxy or amino group, preferably at least one hydroxy group.

Examples are the monohydroxy-functional polyalkylene oxide polyether alcohols having a statistical mean of 5 to 70, preferably 7 to 55, ethylene oxide units per molecule, as are obtainable in a manner known per se by alkoxylation of suitable starter molecules (described, for example, in Ullmanns Encyclopadie der technischen Chemie [Ullmann's Encyclopedia of Industrial Chemistry], 4th edition, volume 19, Verlag Chemie, Weinheim p. 31-38). These compounds are either pure polyethylene oxide ethers or mixed polyalkylene oxide ethers, in which case, however, they contain at least 30 mol %, preferably at least 40 mol %, based on all alkylene oxide units present, of ethylene oxide units. Particularly preferred nonionic compounds are monofunctional mixed polyalkylene oxide polyethers having 40 to 100 mol % of ethylene oxide units and 0 to 60 mol % of propylene oxide units.

For hydrophilization, it is also possible to use mixtures of anionic/potentially anionic hydrophilization agents and nonionic hydrophilization agents

In a development of the invention, the amino-functional chain extender component d) may comprise ≥85 mol %, preferably ≥95 mol %, and more preferably 100 mol %, of IPDA.

In addition to IPDI, further NH₂— and/or NH-functional compounds may be used as further constituents of the amino-functional chain extender d).

The chain extension/termination is preferably carried out before the dispersion in water, with the isocyanate groups reacting with the chain extender to form urea groups.

Suitable components that may be used in addition to IPDA in a molar proportion of less than 25% are di- or polyamines such as ethylene-1,2-diamine, 1,2- and 1,3-diaminopropane, 1,4-diaminobutane, 1,6-diaminohexane, 2,2,4- and 2,4,4-trimethylhexamethylenediamine, 2-methylpentamethylenediamine, diethylenetriamine, triaminononane, xylylene-1,3-diamine and -1,4-diamine, α,α,α′,α′-tetramethylxylylene-1,3-diamine and -1,4-diamine, and 4,4-diaminodicyclohexylmethane and/or dimethylethylenediamine. Likewise possible are hydrazine and/or hydrazides such as adipic dihydrazide.

In addition to components a) to d), other building blocks may also be used in the preparation of the polyurethane urea of the invention.

Examples are hydroxy-functional compounds having molecular weights of 62 to 399 g/mol, for example polyols of the stated molecular weight range having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol. Monofunctional isocyanate-reactive amine compounds may also be used, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine.

It is preferable not to use any further building blocks besides components a) to d) in the preparation of the polyurethane urea for use according to the invention.

The preparation of the polyurethane urea for use thereof according to the invention may be carried out in one or more steps in a homogeneous or multistep reaction according to the methods known to those skilled in the art, and in some cases may be carried out in a disperse phase. After completion of the polyaddition from a) to d) in full or in part, a dispersion, emulsification or dissolution step is preferably carried out. This is optionally followed by a further polyaddition or modification in the disperse or dissolved (homogeneous) phase. Any prior art process may be used, for example the prepolymer mixing process, the acetone process or the melt dispersion process. The acetone process is preferably used.

The invention further provides a method for water-resistant and/or washable shaping of hair into a hairstyle, comprising the steps of:
i. Treating the hair with a polyurethane urea obtainable by reacting at least one polyisocyanate component, a polymeric polyol component, a hydrophilizing component, and an amino-functional chain extender component, wherein the polyisocyanate component a) comprises ≥75 mol % of isophorone diisocyanate (IPDI) and the amino-functional chain extender component c) comprises ≥75 mol % of isophoronediamine (IPDA);
ii. Shaping the hair into the desired hairstyle;
iii. Optionally drying the hair;
iv. Optionally contacting the hair with water.
v. Optionally repeating steps iii. and iv. at least once.

Contacting the hair with water in point iv. is in the context of the invention understood as meaning that at least part of the hair shaped in step ii. is immersed in water. What is to be understood by water has already been defined above. Contacting the hair here preferably includes a washing process with surfactant-containing water, also referred to as shampooing.

In a preferred embodiment of the method, the hair undergoes a washing step before or after each of steps i. to v.

What has been said above with regard to styling retention x_{straightened} for straightened hair and with regard to styling retention x_{curled} for curled hair preferably applies to the method of the invention accordingly.

The invention further provides a water-stable hair cosmetic composition comprising a polyurethane urea obtainable by reacting at least
b) a polyisocyanate component,
c) a polymeric polyol component,
d) a hydrophilizing component, and
e) an amino-functional chain extender component,
   wherein the polyisocyanate component a) comprises ≥75 mol % of isophorone diisocyanate (IPDI) and the amino-functional chain extender component c) comprises ≥75 mol % of isophoronediamine (IPDA) and the composition is in a form selected from the group consisting of a gel, a cream, an aerosol, a spray, a hair wax or a combination of at least two thereof.

The hair cosmetic composition preferably comprises a solvent, a solvent mixture or a dispersant, and a polyurethane urea previously suitable for the use according to the invention, wherein the solvent, the solvent mixture or the dispersant comprises water or ethanol and water. The hair cosmetic composition preferably contains only water as a solvent or dispersant.

The solvent mixture may optionally comprise other cosmetically suitable solvents. Preferred solvents are aliphatic alcohols with C2-4 carbon atoms such as isopropanol, t-butanol, n-butanol; polyols such as propylene glycol, glycerol, ethylene glycol, and polyol ethers; acetone; unbranched or branched hydrocarbons such as pentane, hexane, isopentane, and cyclic hydrocarbons such as cyclopentane and cyclohexane; and mixtures thereof.

In the case of hair cosmetic compositions that contain a solvent mixture, the solvent mixture may comprise ≥10% by weight and ≤98% by weight, preferably ≥15% by weight and ≤98% by weight, more preferably ≥20% by weight and ≤90% by weight, and particularly preferably ≥20% by weight and ≤80% by weight, of ethanol.

It is likewise possible that the solvent mixture consists of water and ethanol.

Preference is given to a composition comprising ≥0.1% by weight and ≤30% by weight, preferably ≥0.1% by weight and ≤20% by weight, more preferably ≥0.5% by weight and ≤15% by weight, and particularly preferably ≥0.5% by weight and ≤10% by weight, of the polyurethane urea.

Preference is also given to a composition comprising ≥10% by weight and ≤98% by weight, preferably ≥20% by weight and ≤98% by weight, more preferably ≥30% by weight and ≤98% by weight, and particularly preferably ≥40% by weight and ≤98% by weight, of the solvent mixture.

In addition to the polyurethane described above, the composition of the invention may comprise further suitable film formers, which in particular may also contribute to the setting and styling of hair.

The proportion of one or more further film formers may be from 0% to 20% by weight and in particular from 0% to 10% by weight, based on the overall formulation.

Advantageously, the further film former(s) is/are selected from the group consisting of nonionic, anionic, amphoteric and/or cationic polymers, and mixtures thereof.

In a preferred embodiment of the hair cosmetic composition, the composition is in a form selected from the group consisting of a pump spray, an aerosol, a gel, a foam, a mousse, a lotion, a wax, a pomade, an oil, a milk, an oil-in-water emulsion, an aqueous solution or a cream. The cosmetic composition is preferably in the form of an oil-in-water, silicone-in-water, water-in-oil, water-in-silicone, oil-in-water-in-oil or water-in-oil-in-water emulsion.

Preference is given to water-in-oil (W/O) or water-in-silicone (W/Si) emulsions that comprise one or more silicone emulsifiers (W/S) having an HLB value 8 or one or more W/O emulsifiers having an HLB value <7 and optionally one or more O/W emulsifiers having an HLB value >10, where HLB (according to Griffin)=20*(1−M₁/M), where M₁ is the molar mass of the lipophilic portion of a molecule and M is the molar mass of the entire molecule.

The silicone emulsifiers may advantageously be selected from the group comprising alkyl dimethicone copolyols, such as cetyl PEG/PPG 10/1 dimethicone copolyol (ABM® EM 90 from Goldschmidt AG) or lauryl PEG/PPG-18/18 dimethicone (Dow Corning® 5200 from Dow Corning Ltd.), and dimethicone copolyols such as PEG-10 dimethicone (KF-6017 from Shin Etsu), PEG/PPG-18/18 dimethicone (Dow Corning 5225C from Dow Corning Ltd.) or PEG/PPG-19/19 dimethicone (Dow Corning BY-11 030 from Dow Corning Ltd.).

W/O emulsifiers having an HLB value <7 may advantageously be selected from the group comprising sorbitan stearate, sorbitan oleate, glyceryl isostearate, polyglyceryl-3-oleate, pentaerythrityl isostearate, methylglucose dioleate, PEG-7 hydrogenated castor oil, polyglyceryl-4 isostearate, hexyl laurate, sorbitan isostearate, polyglyceryl-2 dipolyhydroxystearate, polyglyceryl-diisostearate, PEG-30 dipolyhydroxystearate, diisostearoylpolyglyceryl-3 diisostearate, polyglyceryl-3 dipolyhydroxystearate, polyglyceryl-4 dipolyhydroxystearate, polyglyceryl-3 dioleate, and wool wax alcohol (Eucerit).

O/W emulsifiers having an HLB value >10 may advantageously be selected from the group comprising lecithin, trilaureth-4 phosphate, polysorbate 20, polysorbate 60, PEG-22-dodecyl glycol copolymer, sucrose stearate, and sucrose laurate.

For stabilization of the W/O emulsion of the invention against sedimentation or flocculation of water droplets, an oil thickener may advantageously be used.

Particularly advantageous oil thickeners are organomodified clays such as organomodified bentonites (Bentone® 34 from Rheox), organomodified Hectorite (Bentone® 27 and Bentone® 38 from Rheox) or organomodified montmorillonite, hydrophobic fumed silica, in which the silanol groups are substituted with trimethylsiloxy groups (Aerosil® R812 from Degussa) or with dimethylsiloxy groups or polydimethylsiloxane (Aerosil® R972, Aerosil® R974 from Degussa, Cab-O-Sil® TS-610, Cab-O-Sil® TS-720 from Cabot), magnesium or aluminum stearate, or styrene copolymers such as styrene-butadiene-styrene, styrene-isopropene-styrene, styrene-ethylene/butene-styrene or styrene-ethylene/propene-styrene.

The thickener for the oil phase may be present in an amount of 0.1% to 5% by weight, based on the total weight of the emulsion, and preferably 0.4% to 3% by weight.

The aqueous phase may additionally comprise stabilizing agents. The stabilizing agent may be, for example, sodium chloride, magnesium chloride or magnesium sulfate, and mixtures thereof.

Oils may be used in W/O, W/Si, and O/W emulsions.

If present, the oil phase of the composition of the invention comprises at least one nonvolatile oil. The oil phase of the composition may also contain volatile oils and waxes. The O/W composition advantageously contains 0% to 45% by weight of oils, based on the total weight of the composition, and particularly advantageously 0% to 20% by weight of oils. The W/O or W/Si composition advantageously contains at least 20% by weight of oils, based on the total weight of the composition.

The nonvolatile oil is advantageously chosen from the group consisting of mineral, animal, plant or synthetic origin, polar or nonpolar oils, and mixtures thereof.

Polar oils may be selected from lecithins and fatty acid triglycerides, in particular the triglyceryl esters of saturated and/or unsaturated, branched and/or unbranched alkanecarboxylic acids having a chain length of 8 to 24, in particular 12 to 18, carbon atoms. For example, the fatty acid triglycerides may be chosen from the group consisting of cocoglyceride, olive oil, sunflower oil, soybean oil, groundnut oil, rapeseed oil, almond oil, palm oil, coconut oil, castor oil, wheatgerm oil, grapeseed oil, safflower oil, evening primrose oil, macadamia nut oil, apricot kernel oil, avocado oil, and the like.

Further advantageous polar oils may be selected from the group consisting of esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, and also from the group of esters of aromatic carboxylic acids and saturated and/or unsaturated, branched and/or unbranched alcohols having a chain length of 3 to 30 carbon atoms. For example, the ester oils may preferably be chosen from the group consisting of phenethyl benzoate, octyl palmitate, octyl cocoate, octyl isostearate, octyldodecyl myristate, octyl dodecanol, cetearyl isononanoate, isopropyl myristate, isopropyl palmitate, isopropyl stearate, isopropyl oleate, n-butyl stearate, n-hexyl laurate, n-decyl oleate, diisopropyl adipate, isooctyl stearate, isononyl stearate, isononyl isononanoate, 2-ethylhexyl palmitate, 2-ethylhexyl laurate, 2-hexyldecyl laurate, 2-hexyldecyl stearate, 2-octyldodecyl palmitate, 2-octyldodecyl myristate, 2-octyldodecyl lactate, 2-diethylhexyl succinate, diisostearyl malate, glyceryl triisostearate, diglyceryl triisostearate, stearyl heptanoate, oleyl oleate, oleyl erucate, erucyl oleate, erucyl erucate, tridecyl stearate, tridecyl trimellitate, and also synthetic, semisynthetic, and natural mixtures of such esters, for example jojoba oil.

The polar oils may advantageously be chosen from the group consisting of dialkyl ethers and dialkyl carbonates; advantageous examples are dicaprylyl ether (Cetiol® OE from BASF) and/or dicaprylyl carbonate (for example Cetiol® CC from BASF).

It is further preferable that the polar oils are selected from the group consisting of isoeicosane, neopentyl glycol diheptanoate, propylene glycol dicaprylate/dicaprate, caprylic/capric/diglyceryl succinate, butylene glycol dicaprylate/dicaprate, C₁₂₋₁₃ alkyl lactate, di-C₁₂₋₁₃ alkyl tartrate, C12-15 alkyl benzoate, myristyl myristate, isodecyl neopentanoate, triisostearin, dipentaerythrityl hexacaprylate/hexacaprate, propylene glycol monoisostearate, tricaprylin, dimethyl isosorbide, butyloctyl salicylate (as obtainable for example under the trade name Hallbrite® BHB from CP Hall), hexadecyl benzoate and butyloctyl benzoate and mixtures thereof (Hallstar® AB) and/or diethylhexyl naphthalate (Hallbrite® TQ or Corapan® TQ from Symrise).

The nonvolatile oil may likewise advantageously also be a nonpolar oil chosen from the group consisting of branched and unbranched hydrocarbons, in particular mineral oil, vaseline oil, paraffin oil, squalane and squalene, polyolefins, for example polydecenes, hydrogenated polyisobutenes, C13-16 isoparaffin, and isohexadecane.

The nonpolar nonvolatile oil may be selected from the nonvolatile silicone oils.

The nonvolatile silicone oils may include polydimethylsiloxanes (PDMS) that are optionally phenylated, such as phenyltrimethicone, or are optionally substituted by aliphatic and/or aromatic groups or by functional groups, for example hydroxy groups, thiol groups and/or amino groups; polysiloxanes modified with fatty acids, fatty alcohols or polyoxyalkylenes, and mixtures thereof.

The composition of the invention may further comprise a wax.

In the context of the present document, a wax is defined as a lipophilic fatty substance that is solid at room temperature (25° C.) and shows a reversible solid/liquid change of state at a melting temperature between 30° C. and 200° C. Above the melting point, the viscosity of the wax is low and it becomes miscible with oils.

The wax is advantageously chosen from the groups of natural waxes, for example cotton wax, carnauba wax, candelilla wax, esparto wax, japan wax, montan wax, sugarcane wax, beeswax, wool wax, shellac, microwaxes, ceresin, ozokerite, ouricury wax, cork fiber wax, lignite waxes, berry wax, shea butter, or synthetic waxes such as paraffin waxes, polyethylene waxes, waxes produced by Fischer-Tropsch synthesis, hydrogenated oils, fatty acid esters and glycerides that are solid at 25° C., silicone waxes and derivatives (alkyl derivatives, alkoxy derivatives and/or esters of polymethylsiloxane) and mixtures thereof. The waxes may take the form of stable dispersions of colloidal wax particles, which can be produced by known methods, for example according to “Microemulsions Theory and Practice”, L.M. Prince Ed., Academic Press (1977), pages 21-32.

The waxes may be present in amounts of 0% to 10% by weight, based on the total weight of the composition, and preferably 0% to 5% by weight.

The composition of the invention may further comprise a volatile oil selected from the group consisting of volatile hydrocarbons, siliconized oils, and fluorinated oils.

The volatile oil may be present in an amount of 0% to 25% by weight, based on the total weight of the emulsion, preferably 0% to 20% by weight, and more preferably 0% to 15% by weight.

In the context of the present document, a volatile oil is an oil that evaporates within less than one hour on contact with skin at room temperature and atmospheric pressure. The volatile oil is liquid at room temperature and, at room temperature and atmospheric pressure, has a vapor pressure of 0.13 to 40 000 Pa (10 to 300 mm Hg), preferably 1.3 to 13 000 Pa (0.01 to 100 mm Hg), and more preferably 1.3 to 1300 Pa (0.01 to 10 mm Hg), and a boiling point of 150 to 260° C. and preferably 170 to 250° C.

A hydrocarbon oil is understood as meaning an oil that is formed largely from carbon atoms and hydrogen atoms, with or without oxygen atoms or nitrogen atoms, and does not contain any silicon atoms or fluorine atoms; it may also consist of carbon atoms and hydrogen atoms, but it may also contain ester groups, ether groups, amino groups or amide groups.

A silicone oil is understood as meaning an oil containing at least one silicon atom and especially

Si—O groups, such as polydiorganosiloxanes in particular.

A fluorinated oil is understood as meaning an oil containing at least one fluorine atom. The volatile hydrocarbon oil of the invention may be selected from the hydrocarbon oils having a flash point of 40 to 102° C., preferably 40 to 55° C., and more preferably 40 to 50° C.

The volatile hydrocarbon oils are preferably those having 8 to 16 carbon atoms and mixtures thereof, in particular branched C₈₋₁₆ alkanes such as isoalkanes (also referred to as isoparaffins) having 8 to 16 carbon atoms, isododecane, isodecane, isohexadecane and, for example, the oils marketed under the Isopars® or Permetyls® trade names; and the branched C₈₋₁₆ esters, such as isohexyl neopentanoate, and mixtures thereof.

Particularly advantageous are volatile hydrocarbon oils such as isododecane, isodecane, and isohexadecane.

The volatile siliconized oil is preferably selected from siliconized oils having a flash point of 40 to 102° C., preferably a flash point exceeding 55° C. and not more than 95° C., and more preferably in the range from 65 to 95° C.

For example, the volatile siliconized oils are straight-chain or cyclic silicone oils having 2 to 7 silicon atoms, these silicones optionally containing alkyl or alkoxy groups having 1 to 10 carbon atoms.

Particularly advantageous are volatile siliconized oils such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

The volatile fluorinated oil does not generally have a flash point.

Examples of volatile fluorinated oils are nonafluoroethoxybutane, nonafluoromethoxybutane, decafluoropentane, tetradecafluorohexane, dodecafluoropentane, and mixtures thereof.

The preferred cosmetically acceptable medium of the composition of the invention comprises water and optionally a cosmetically acceptable water-miscible suitable organic solvent.

The water used in the composition of the invention may be a blossom water, pure demineralized water, mineral water, thermal water, and/or seawater.

In the case of an O/W composition as the composition of the invention, the water content may be in the range from 40% to 95% by weight, preferably in the range from 50% to 90% by weight, most preferably in the range from 60% to 80% by weight, based on the total weight of the composition.

In the case of a W/O composition, the water content is in the range from 0% to 60% by weight, preferably in the range from 10% to 50% by weight, most preferably in the range from 30% to 50% by weight, based on the total weight of the composition.

The composition may also be foamed with a propellant gas. The above-described emulsions may be stabilized by O/W, W/O or W/Si emulsifiers, thickeners (such as hydrodispersion) or solids (for example a Pickering emulsion).

The composition may contain one or more emulsifiers or surface-active agents.

Thus, oil-in-water emulsions (O/W) in particular preferably contain at least one emulsifier having an HLB value >7 and optionally a coemulsifier.

The following nonionic emulsifiers are used advantageously:
a) fatty acid partial esters and fatty acid esters of polyhydric alcohols and ethoxylated derivatives thereof (for example glyceryl monostearate, sorbitan stearate, glyceryl stearyl citrate, sucrose stearate)
b) ethoxylated fatty alcohols and fatty acids.

Particularly advantageous nonionic O/W emulsifiers are ethoxylated fatty alcohols or fatty acids, preferably PEG-100 stearate, PEG-40 stearate, ceteareth-20, ceteth-20, steareth-20, ceteareth-12, ceteth-12, steareth-12 and esters of mono-, oligo- or polysaccharides with fatty acids, preferably cetearyl glucoside, methyl glucose distearate.

Advantageous anionic emulsifiers are soaps (for example sodium or triethanolamine salts of stearic acid or palmitic acid) and esters of citric acid such as glyceryl stearate citrate.

Suitable coemulsifiers used for O/W emulsions of the invention may be fatty alcohols having 8 to 30 carbon atoms, monoglyceryl esters of saturated or unsaturated, branched or unbranched alkanecarboxylic acids having a chain length of 8 to 24 carbon atoms, especially 12 to 18 carbon atoms, propylene glycol esters of saturated or unsaturated, branched or unbranched alkanecarboxylic acids having a chain length of 8 to 24 carbon atoms, especially 12 to 18 carbon atoms, and sorbitan esters of saturated or unsaturated, branched or unbranched alkanecarboxylic acids having a chain length of 8 to 24 carbon atoms, especially 12 to 18 carbon atoms.

Particularly advantageous coemulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol, and polyethylene glycol (2) stearyl ether (steareth-2).

It may be advantageous in the context of the present invention to use further emulsifiers. This may be done, for example, to further increase the water resistance of the formulations of the invention.

Examples of suitable emulsifiers are alkyl methicone copolyols and alkyl dimethicone copolyols, in particular cetyl dimethicone copolyol, lauryl methicone copolyol, W/O emulsifiers such as sorbitan stearate, glyceryl stearate, glycerol stearate, sorbitan oleate, lecithin, glyceryl isostearate, polyglyceryl-3 oleate, polyglyceryl-3 diisostearate, PEG-7 hydrogenated castor oil, polyglyceryl-4 isostearate, acrylate/C₁₀₋₃₀ alkyl acrylate crosspolymer, sorbitan isostearate, poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoylpolyglyceryl-3 diisostearate, glycol distearate, and polyglyceryl-3 dipolyhydroxystearate.

What has been said above with regard to styling retention x_{straightened} for straightened hair and with regard to styling retention x_{curled} for curled hair preferably applies to the composition of the invention accordingly.

Preparation (Curling or Straightening) of Hair Tresses (Step 0)):

The tests were carried out using either wavy tresses of European hair (overall length 18 cm, width 8 cm, weight 1.0 g±0.2 g) or straight tresses of European hair (overall length 19 cm, width 3 cm, weight 1.0 g±0.2 g).

Prior to testing, the hair tresses were washed with 0.3 g of a commercial silicone-free shampoo (Syoss “Volume Lift Shampoo”) at 38° 5 C for 1 minute. The hair tresses were then rinsed at 38° C. for 1 minute and combed through with a conventional comb (broad side). The hair tresses were then dried with a hairdryer for 1 minute at about 75° C.

Substances Used

The substances tested are abbreviated as follows:

Product A: INCI: Polyurethane-48, 2% by weight [inventive]

Product B: Amphomer (INCI: Octylacrylamide/Acrylates/Butylaminoethyl Methacrylate Copolymer), 100% with AMP (Amino Methylpropanol) adjusted to pH 9.6, 2% by weight.

Product C: Luviskol PVP K90, INCI: PVP (polyvinylpyrrolidone), 2% by weight.

Product D: INCI: Polyurethane-35, 2% by weight.

Product E: Commercial hair gel, Henkel Got2B glue

Product F: Commercial hair gel, L'Oreal Studio Line Invisi'Hold FX 8

### Example 1: Water Resistance of Straightened Hair

Step 1)

### Comparative Example 1

After preparation as described in step 0), the hair tresses were dried again with a hairdryer for 3 minutes at 75° C. After this, the hair tresses were straightened 5 times for 3 seconds at 230° C. using a commercially available straightening iron and then completely dried for 12 hours at 56% relative-25 air humidity and 23° C.

### Example 1A and 1B: Treatment with Product A or Product B

3 hair tresses were treated identically in parallel. After preparation of the hair tresses as described in step 0), 1 g of product A or B was in each case applied to the respective hair tress. Each of the three hair tresses was run through briefly to distribute the product along the length of the respective hair tress. The hair tresses were combed and dried with a hairdryer for 3 minutes at 75° C. After this, they were straightened with a straightening iron 5 times for 3 seconds at 230° C. and then completely dried for 12 hours at 56% air humidity and 23° C.

### Example 2: Water Resistance of Curled Hair

### Comparative Example 2

After preparation as described in step 0), the hair tresses were twisted on a perm rod and fastened with elastic bands. The tresses were then dried with a hairdryer for 5 minutes at 75° C. and then completely dried for 12 hours at 56% relative-25 air humidity and 23° C.

### Example 2, A to F: Treatment with Products A to F

After preparation of the hair tresses as described in step 0), 1 g of product A, B, C, D, E or F was in each case applied to the respective hair tress. Each of the three hair tresses was run through briefly to distribute the product along the length of the respective hair tress. The hair tresses were combed and twisted on a perm rod and fastened with elastic bands. The tresses were then dried with a hairdryer for 5 minutes at 75° C. and then completely dried for 12 hours at 56% relative-25 air humidity and 23° C.

Water-Resistance Test

Each of the hair tresses treated in examples 1 and 2 was individually fully immersed for 30 seconds in a 2-liter water bath heated to 38° C., allowed to drip for 10 seconds, and allowed to dry completely at room temperature in air for 24 h.

The width and length of the tress was then measured with a ruler (1 millimeter scale).

### Results of Example 1

| | | Width | Width after | |
|---|---|---|---|---|
| | | before/after | water-resistance | Styling |
| | Product | straightening | test = test | retention |
| Example | used | [cm] | width [cm] | [%] |
| 1-comparative | — | 8/1.4 | 5 | 45.45 |
| example | | | | |
| 1A | A | 8 /1.4 | 1.8 | 93.9 |
| 1B | B | 8 /1.4 | 2.8 | 78.7 |

$Styling retention x = \frac{100 * [ Width before straightening - test width ]}{[ \begin{matrix}Width before straightening - \\ width after straightening\end{matrix} ]}$

### Results of Example 2

| | | | Curl length | | | |
|---|---|---|---|---|---|---|
| | | | after the | | | |
| | | | water-resistance | | | |
| | | Length | test | | | |
| | | before/ | [cm] | | | |
| | | after | | Dry = | Differ- | Reten- |
| | Product | rolling | | test | ence* | tion |
| Example | used | [cm] | Wet | length | [cm] | [%] |
| 2-comparative | No | 19/6.5 | 19 | 19 | 12.5 | 0 |
| example | polymer | | | | | |
| 2A (inventive) | A | 19/7.5 | 16.5 | 17.5 | 10 | 13 |
| 2B (comparison) | B | 19/5.5 | 18.5 | 18.5 | 13 | 3.7 |
| 2C (comparison) | C | 19/7.0 | 19 | 19 | 12 | 0 |
| 2D (comparison) | D | 19/6.5 | 18.5 | 18.5 | 12 | 4 |
| 2E (comparison) | E | 19/7.0 | 16.5 | 18.5 | 11.5 | 4.1 |
| 2F (comparison) | F | 19/7.5 | 18.5 | 19 | 11.5 | 0 |

*Difference = Length after water-resistance test, dry (test length) − Length immediately after rolling
$Styling retention x = \frac{100 * [ Length before rolling - test length ]}{[ Length before rolling - length after rolling ]}$

The values in tables 1 and 2 show clearly that treating hair with the hair cosmetic composition of the invention, which contains the special polyurethane urea, leads to an improvement in the washability and water resistance of hairstyles shaped with the polyurethane urea. The recorded styling retention levels are thus markedly lower compared to no addition of polymer, but also to conventional agents such as those used in comparative tests B to F. This is particularly striking in the case of maintaining curl in the hair, since, without using a styling agent or through the use of conventional agents, either no water resistance was demonstrated or water resistance was only one third as high as it was with the hair cosmetic composition of the invention or with use according to the invention of the described polyurethane urea.

1. A hair cosmetic composition for water-stable styling of hair, comprising a polyurethane urea obtained by reacting at least
a) a polyisocyanate component,
b) a polymeric polyol component,
c) a hydrophilizing component, and
d) an amino-functional chain extender component,
wherein the polyisocyanate component a) comprises ≥75 mol % of isophorone diisocyanate and the amino-functional chain extender component c) comprises ≥75 mol % of isophoronediamine.

2. (canceled)

3. (canceled)

4. (canceled)

5. (canceled)

6. The composition as claimed in claim 1, wherein the hair cosmetic composition is selected from the group consisting of a gel, a cream, an aerosol, a spray, a hair wax, and a combination of at least two thereof.

7. The composition as claimed in claim 1, wherein the polyisocyanate component a) comprises ≥80 mol % of isophorone diisocyanate.

8. The composition as claimed in claim 1, wherein the polymeric polyol component b) has a number-average molecular weight of ≥400 and ≤8000 g/mol and/or a mean OH functionality of 1.5 to 6.

9. The composition as claimed in claim 1, wherein the polymeric polyol component b) comprises a polyester.

10. The composition as claimed in claim 1, wherein the hydrophilizing component c) comprises an anionically hydrophilizing component.

11. The composition as claimed in claim 1, wherein the amino-functional chain extender component d) comprises ≥85 mol % of isophoronediamine.

12. (canceled)

13. (canceled)

14. A method for water-resistant and/or washable shaping of hair into a hairstyle, comprising:
i. Treating the hair with a polyurethane urea obtained by reacting at least one polyisocyanate component, a polymeric polyol component, a hydrophilizing component, and an amino-functional chain extender component, wherein the polyisocyanate component comprises ≥75 mol % of isophorone diisocyanate and the amino-functional chain extender component comprises ≥75 mol % of isophoronediamine;
ii. Shaping the hair into tea desired hairstyle;
iii. Optionally drying the hair;
iv. Optionally contacting the hair with water.
v. Optionally repeating steps iii. and iv. at least once.

15. The method as claimed in claim 14, wherein the hair undergoes a washing step before or after each of steps i. to v.

16. (canceled)

17. The method as claimed in claim 14, wherein the shaping of the hair into the desired hairstyle comprises at least one of:
a. straightening curly hair;
b. introducing curls into straight hair;
c. strengthening curls in already curled hair; or
d. a combination of at least two of a. to c.

18. The method as claimed in claim 17, wherein the straightening of the hair under a. is at least 50% retained after contact with water or curling introduced to the hair under b. is at least 30% retained or strengthening of curls under c. is at least 20% retained.

19. The method as claimed in claim 14, wherein a shape of styled hair is at least 5% retained after contact with water.

20. The method as claimed in claim 14, wherein a styling retention x_{straightened} for straightened hair is at least 50% after contact with water.

21. The method as claimed in claim 14, wherein a styling retention x_{curled} for curled hair is at least 5%.
